(19) United States
(12) Patent Application Publication
(10) Pub. No.: US 2020/0268514 A1
(43) Pub. Date: Aug. 27, 2020

(54) MECHANICALLY LOCKING ADJUSTABLE CARDIAC TETHER

(71) Applicant: ARGO CARDIOVASCULAR, INC., Lebanon, NH (US)

(72) Inventor: Stephen KAO, Sunnyvale, CA (US)

(21) Appl. No.: 16/789,250

(22) Filed: Feb. 12, 2020

Related U.S. Application Data
(60) Provisional application No. 62/809,116, filed on Feb. 22, 2019.

(51) Int. Cl. A61F 2/24

(52) U.S. Cl. CPC A61F 2/2487; A61F 2002/249; A61F 2220/0041; A61F 2220/0016

(57) ABSTRACT

A method of modifying a shape of a patient's heart by placing a first anchor against a surface of a first wall of the heart; extending a tether proximally from the first anchor to a second wall of the heart; placing a second anchor against a surface of the second wall; rotating a nut about the tether to move the second anchor distally along the tether, thereby moving the second wall toward the first wall; and permitting the nut to remain in place after the rotating step. The invention also includes a device for practicing the method.

### CROSS REFERENCE TO RELATED APPLICATIONS

This application claims the benefit of priority to U.S. Provisional Application No. 62/809,116, filed Feb. 22, 2019, which application is incorporated by reference herein.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Ischemic and Non Ischemic Dilated Cardiomyopathy causes the heart to become enlarged and to function poorly. Some people have stable disease and there is little worsening of their condition. Others have progressive disease. As a result, the muscle of the heart becomes weak, thin or floppy and is unable to pump blood efficiently around the body. This inability to efficiently pump blood typically causes fluid to build up in the lungs, which then become congested, resulting in a feeling of breathlessness. This condition is referred to as congestive (left) heart failure. Often there is also right heart failure which causes fluid to accumulate in the tissues and organs of the body, usually the legs and ankles, and the liver and abdomen. Left ventricular dilation can also lead to secondary Mitral valvular regurgitation, further worsening cardiac performance, referred to as functional Mitral Regurgitation.

The typical pathology of Dilated Cardiomyopathy includes dilation of the ventricle and contraction deficiency, and heart failure systems appear in 75 to 95% of patients, often with complications of arrhythmic-death (sudden death) or thrombosis and embolism during the course of the disease. It is an intractable disease with a mortality rate of approximately 50% within 5 years of onset. This disease also accounts for the majority of heart transplant patients in Europe and the United States.

In Dilated Cardiomyopathy, the size of the valve annulus (particularly the mitral valve annulus) increases while the area of the leaflets of the valve remains constant. This may lead to an area of less coaptation of the valve leaflets, and, as a result, eventually to valve leakage. Moreover, in normal hearts, the annular size contracts during systole, aiding in valve coaptation. In heart failure, there is poor ventricular function and elevated wall stress. These effects tend to reduce annular contraction and distort annular size, often exacerbating mitral valve regurgitation. In addition, as the chamber dilates, the papillary muscles (to which the leaflets are connected via the chordae tendonae) may move radially outward and downward relative to the valve, and relative to their normal positions. During this movement of the papillary muscles, however, the various chordae lengths remain substantially constant, which limits the full closure ability of the leaflets by exerting tension prematurely on the leaflets. This condition is commonly referred to as “chordal tethering.” The combination of annular changes and papillary changes results in a poorly functioning valve.

It has been observed that for at least certain locations and orientations of the one or more native transventricular tethers in humans, a pre-existing mitral valve incompetency can be exacerbated by the presence and impact of the tethers. The tether and the local deformation they impart may further alter the positions of the papillary muscles in such a way that the chordae do not allow as complete of a closure of the mitral valve, or that rotation of portions of the ventricular wall (to which additional chordae may be attached) may “tighten” one valve leaflet and “loosen” the other. In this manner, the leaflets may not close at the same level relative to the annulus, causing increased retrograde leakage through the valve.

Even in instances where the placement of tethers does not contribute to further mitral valve leakage, it may be desirable to provide a therapy which could also correct the valve incompetency. A heart with even a small amount of regurgitation may benefit from not only the stress reducing functions of the ventricular tether as described above, but also from the elimination of the regurgitation, which will further off-load the pumping requirements of the myocardium.

While currently available methods of mitral valve repair or replacement are possible to employ in conjunction with ventricular tethering, they typically require opening the heart to gain direct access to the valve and its annulus. This type of access necessitates the use of cardiopulmonary bypass or open heart access, which can introduce additional complications to the surgical procedure. Since the implantation of the tethers themselves do not require the patient to be on cardiopulmonary bypass or to have open heart access, it would be advantageous to devise a technique which could improve the ventricle and mitral valve without the need for these surgeries. The ability to improve the ventricular dilation and mitral valve function without the need for cardiopulmonary bypass or open heart access would be an advantage, both in conjunction with ventricular tethering, and also as a stand-alone therapy.

### SUMMARY OF THE DISCLOSURE

Objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims. To achieve the objects and in accordance with the purpose of the invention, as embodied and broadly described herein, one aspect of the invention comprises a method for improving the function of a ventricle and valve of a heart.

The method includes the steps of placing an elongate member transverse a heart chamber so that a first end of the elongate member extends through a wall of the heart, and a second end of the elongate member extends through a septum of the heart; placing a first anchoring member external the heart; and placing a second anchoring member inside the heart adjacent the septum. The first and second anchoring members are attached to first and second ends of the elongate member respectively to fix the elongate member in a position across the chamber. The anchors are mechanically repositioned by adjusting the position of one anchoring member to shorten the effective length of the elongate member. After the appropriate anchor adjustment is performed, the ends of the elongate member are permanently fixed so as to decrease the ventricular diameter, alter the cross-sectional shape of the ventricle, and assist coaptation of valve leaflets, thus improving cardiac function.

According to another aspect, the invention comprises a tether for improving the function of a ventricle and a valve of a heart. The tether includes an elongate member configured to be positioned transverse a heart chamber so that a first end of the elongate member extends through a wall of the heart, and a second end of the elongate member extends through a septum of the heart; placing a first anchoring member external the heart; and placing a second anchoring member inside the heart adjacent the septum. The first and second anchoring members are attached to first and second ends of the elongate member respectively to fix the elongate member in a position across the chamber. The anchors are mechanically repositioned by adjusting the position of one anchoring member to shorten the effective length of the elongate member. After the appropriate anchor adjustment is performed, the ends of the elongate member are permanently fixed and the excess elongate member is cut and removed. The invention decreases the ventricular diameter, alters the cross-sectional shape of the ventricle, and assists coaptation of valve leaflets, thus improving cardiac function. Adjustment of the anchor position and tether length can occur simultaneously with real-time monitoring of cardiac function using echocardiographic and/or fluoroscopic visualization of ventricular shape and size and valve coaptation.

It is to be understood that both the foregoing general description and the following detailed description are exemplary and explanatory only and are not restrictive of the invention, as claimed.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a partial vertical cross-sectional view of the lower half of the heart, specifically the left ventricle (LV), right ventricle (RV), free wall, and septal wall.

FIG. 2 is a partial vertical cross-sectional view of the lower half of the heart showing a puncture needle or guide wire inserted endovascularly through the free wall first and then through the septal wall.

FIG. 3 is a partial vertical cross-sectional view of the lower half of the heart showing a tether system inserted endovascularly through the free wall, across the left ventricle, and through the septal wall over the guidewire or replacing the puncture needle according to an aspect of the present invention.

FIG. 4 is a partial vertical cross-sectional view of the lower half of the heart showing the sheath of a tether system being retracted and the first anchor being deployed (or expanded) and positioned adjacent to the septal wall in the right ventricle.

FIG. 5 is a partial vertical cross-sectional view of the lower half of the heart showing the sheath of a tether system being further retracted and the second anchor being deployed (or expanded) and positioned adjacent to the free wall outside of the heart.

FIG. 6 is a partial vertical cross-sectional view of the lower half of the heart showing the nut driver/cutter being rotated to advance itself and the locking nut to be positioned adjacent to the second anchor.

FIG. 7 is a partial vertical cross-sectional view of the lower half of the heart showing the nut driver/cutter being further rotated to advance itself, the locking nut, and second anchor towards the first anchor. The free wall is compressed and the left ventricle diameter is reduced.

FIG. 8 is a partial vertical cross-sectional view of the lower half of the heart showing the microblade at the distal end of the nut driver/cutter being actuated to cut the tether proximal of the locking nut. The tether and anchors are no longer connected to the delivery system.

FIG. 9 is a partial vertical cross-sectional view of the lower half of the heart showing the delivery system is removed and the tether implant maintaining reduction of the left ventricle.

FIG. 10 is a vertical cross-sectional view of the heart showing a delivery catheter inserted endovascularly into the right ventricle according to an aspect of the present invention.

FIG. 11 is a vertical cross-sectional view of the heart showing a guide wire extending from the delivery catheter through the septal wall, across the left ventricle, and into the free wall according to an aspect of the present invention.

FIG. 12 is a vertical cross-sectional view of the heart showing a tether system inserted endovascularly through the septal wall, across the left ventricle, and through the free wall over the guide wire insertion according to an aspect of the present invention.

FIG. 13 is a vertical cross-sectional view of the heart showing the sheath of a tether system being retracted and the first anchor being deployed (or expanded) and positioned adjacent to the free wall outside the heart.

FIG. 14 is a vertical cross-sectional view of the heart showing the sheath of a tether system being further retracted and the second anchor being deployed (or expanded) and positioned adjacent to the septal wall in the right ventricle.

FIG. 15 is a vertical cross-sectional view of the heart showing the nut driver/cutter rotated to advance itself, the locking nut, and second anchor towards the first anchor. The septal wall is compressed and the left ventricle diameter is reduced.

FIG. 16 is a vertical cross-sectional view of the heart showing the microblade at the distal end of the nut driver/cutter having cut the tether proximal of the locking nut. The tether and anchors are no longer connected to the delivery system. The delivery system is removed and the tether implant maintains reduction of the left ventricle.

FIG. 17 is a top cross-sectional view of the heart showing the microblade at the distal end of the nut driver/cutter having cut the tether proximal of the locking nut. The tether and anchors are no longer connected to the delivery system. The delivery system is removed and the tether implant maintains reduction of the left ventricle.

FIGS. 18A-C show an anchor with a braided wire structure. It may have one (FIG. 18A) or two (FIG. 18B) braided discs, whereby each disc would be adjacent to both sides of the ventricular wall. FIG. 18C shows an end view.

FIGS. 19A and 19B show anchors with a laser cut tubular shape set structure.

### DETAILED DESCRIPTION

The various aspects of the invention to be discussed herein generally pertain to devices and methods for treating heart conditions, including, for example, dilatation, valve incompetencies, including mitral valve leakage, and other similar heart failure conditions. Each device of the present invention preferably operates passively in that, once placed in the heart, it does not require an active stimulus, either mechanical, electrical, or otherwise, to function. Implanting one or more of these devices alters the shape or geometry of the heart, both locally and globally, and thereby increases the heart's efficiency. That is, the heart experiences an increased pumping efficiency through an alteration in its shape or geometry and concomitant reduction in stress on the heart walls. In addition, the devices of the present invention may operate to assist in the apposition of heart valve leaflets to improve valve function.

The inventive devices and related methods offer numerous advantages over the existing treatments for various heart conditions, including valve incompetencies. The devices are relatively easy to manufacture and use, and the surgical techniques and tools for implanting the devices of the present invention do not require the invasive procedures of current surgical techniques. For instance, the endovascular techniques which will be described do not require performing a sternotomy or removing portions of the heart tissue, nor do they require opening the heart chamber or stopping the heart during operation. Such percutaneous insertion permits the splinting procedures to be performed in a wide variety of laboratories in the hospital. For these reasons, the techniques for implanting the devices of the present invention also are less risky to the patient, both during and after the implantation, and may be performed more quickly than other techniques. For instance, the procedures of the invention cause less pain to patients and permit quicker healing. In addition, certain endovascular splinting techniques to be described may limit bleeding at access sites, allowing relatively large catheters, cannula, and other similar implantation tools to be inserted in a percutaneous manner.

The disclosed inventive devices and related methods involve geometric reshaping of the heart. In certain aspects of the inventive devices and related methods, substantially the entire chamber geometry is altered to return the heart to a more normal state of stress. Models of this geometric reshaping, which includes a reduction in radius of curvature of the chamber walls, can be found in U.S. Pat. No. 5,961,440, issued Oct. 5, 1999, entitled “Heart Wall Tension Reduction Apparatus and Method,” and incorporated by reference herein. Prior to reshaping the chamber geometry, the heart walls experience high stress due to a combination of both the relatively large increased diameter of the chamber and the thinning of the chamber wall. Filling pressures and systolic pressures are typically high as well, further increasing wall stress. Geometric reshaping according to the present invention reduces the stress in the walls of the heart chamber to increase the heart's pumping efficiency, as well as to stop further dilatation of the heart.

It also is contemplated that the inventive endovascular splinting devices and methods will be used to support an infarcted heart wall to prevent further dilatation, or to treat aneurysms in the heart. U.S. Pat. No. 6,406,420, issued Jun. 18, 2002, entitled “Methods and Devices for Improving Cardiac Function in Hearts,” discusses this form of heart failure in more detail. Moreover, it is contemplated that the devices and methods of using and implanting the devices could be used to treat heart valves, for example to aid in apposition of the leaflets of a mitral valve or modify the shape of the mitral valve. One of ordinary skill in the art would understand that the use of the devices and methods described herein also could be employed in other chambers and for other valves associated with those chambers. For example, the devices and methods of the invention might be used to reduce stress in the left atrium to treat atrial fibrillation. The left ventricle has been selected for illustrative purposes because a large number of the disorders that the present invention treats occur in the left ventricle.

Reference will now be made in detail to the present preferred embodiments of the invention, examples of which are illustrated in the accompanying drawings. Wherever possible, the same reference numbers will be used throughout the drawings to refer to the same or like parts.

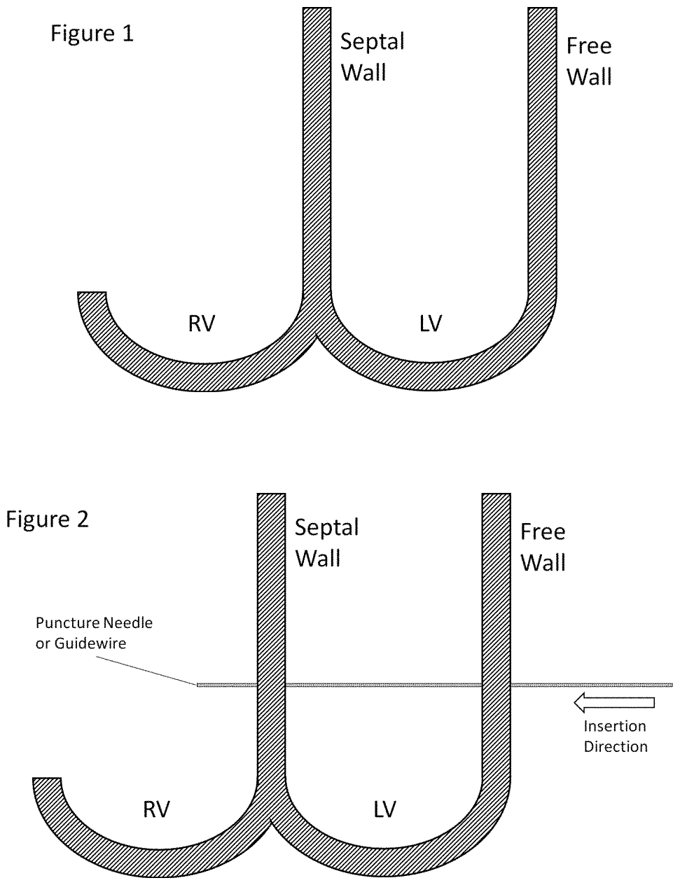

An embodiment of an endovascular tethering technique according to the present invention is shown in FIGS. 1-9. In this tethering technique, access to the left ventricle LV and delivery of the tether occurs from outside the heart. This surgical technique has an ease of approach and applicability to other surgical procedures involving the heart, i.e. thoracotomy, open-heart surgery. Because access is via the surface of the chest, the procedure is in close proximity to the physician and the working length of the procedure is short.

Standard access to the heart may be performed prior to insertion of the present invention to the left ventricle. Minimally invasive access may also be possible with appropriate imaging guidance and the proper navigation and delivery of the device through the heart. Performing less invasive surgical procedures thereby minimizes trauma to the patient. Additionally, patients are less likely to experience embolic events. Recovery times for the operation also are reduced.

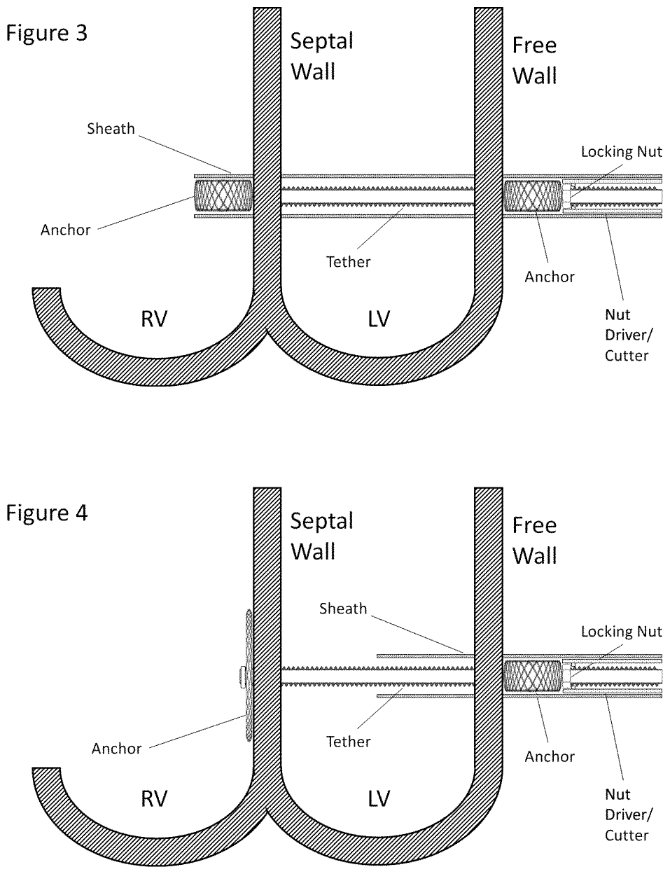

According to the present invention shown in FIG. 3, the tether delivery system comprises a first anchor (shown in the right ventricle RV in FIG. 3), a tether, a second anchor (shown outside of the left ventricle LV in FIG. 3), a sheath, a locking nut, and a nut driver/cutter. The tether assembly which is implanted into the heart from the delivery system comprises the anchors and the tether. The tether is an elongate member connecting the anchors whereby the first end of the elongate member is attached to the first anchor and the second end of the elongate member is attached to the second anchor. The tether assembly is constrained inside the sheath with the locking nut and nut driver/cutter proximal to the assembly.

In the illustrated embodiment, the tether delivery system comprising a sheath containing the anchors, tether and other components is inserted into the heart over a puncture needle or guidewire that extends through the left ventricle LV into the right ventricle RV, as shown in FIGS. 2 and 3. Deployment of the tether assembly is performed by retracting the sheath relative to the other components of the system. The first anchor self-expands to be deployed in the right ventricle and can be repositioned adjacent to the septal wall after it has fully expanded outside of the sheath of the delivery system.

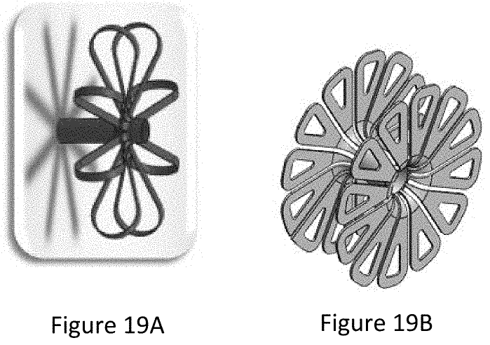

The first anchor can be fabricated from Nitinol, Elgiloy, a shape memory alloy (SMA), a shape memory polymer (SMP), or another material that enables the expansion of the anchor in the ventricle from its original constrained state inside the delivery system sheath. The anchor can be a braided wire structure as shown in FIG. 18. It may have one or two braided discs, whereby each disc would be adjacent to both sides of the ventricular wall. The anchor can also be a laser cut tubular shape set structure as shown FIG. 19A or FIG. 19B.

One embodiment of the laser cut anchor is shaped like a flower with multiple (8 or more) struts in order to maximize the surface area of the anchor in contact with the ventricular wall and optimally distribute the load from the anchor to the tissue.

A patch of material from Polyethylene or PTFE or another biocompatible polymer may be woven into the anchor structure to provide hemostasis and minimize blood flow in and out of the left ventricle through the anchors.

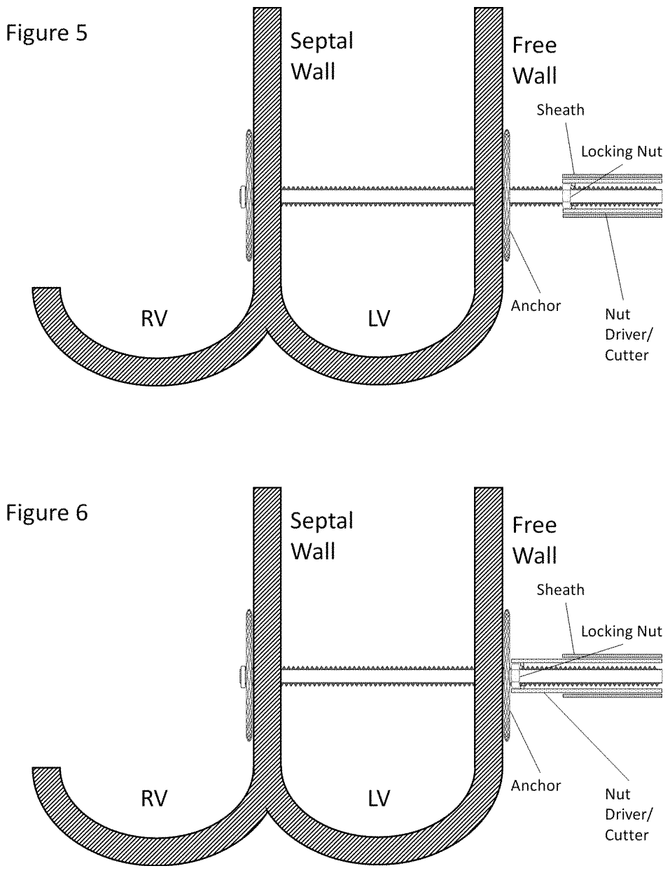

The sheath is further retracted to allow the second anchor to self-expand and deploy outside of the free wall of the heart as shown in FIG. 5. The second anchor can be constructed from the same material and have the same design as the first anchor. The second anchor can be re-positioned adjacent or close to the free wall after it has fully expanded outside of the sheath of the delivery system.

An engagement surface of the nut driver/cutter is engaged with the locking nut in a predetermined fashion and can be rotated to advance both itself and the locking nut in the distal direction over the tether until the locking nut contacts the second anchor as shown in FIG. 6. The engagement surface of the nut driver/cutter and the locking nut have mating shapes, sizes, and features (if present) that enable them to engage and disengage when necessary. One embodiment of the locking nut is a hexagonal nut with a pitch between 56 to 90 threads per inch.

The nut driver/cutter and locking nut are advanced over the tether which has external threads with a corresponding pitch to the locking nut. The tether can be either tubular, or a rod or filament. The tether may be made from PET or Nylon material or a high strength polymer and tubular in design to allow for delivery over a guide wire. The pitch of the tether may be higher or lower to obtain the desired travel per rotation of the nut driver/cutter. Portions of the tether may or may not be threaded. One embodiment of the tether is threaded only in the section over which the locking nut travels. The portion of the tether that is in the left ventricle that is not contacted by the locking nut may remain as extruded with a smooth surface to promote biocompatibility and hemocompatibility.

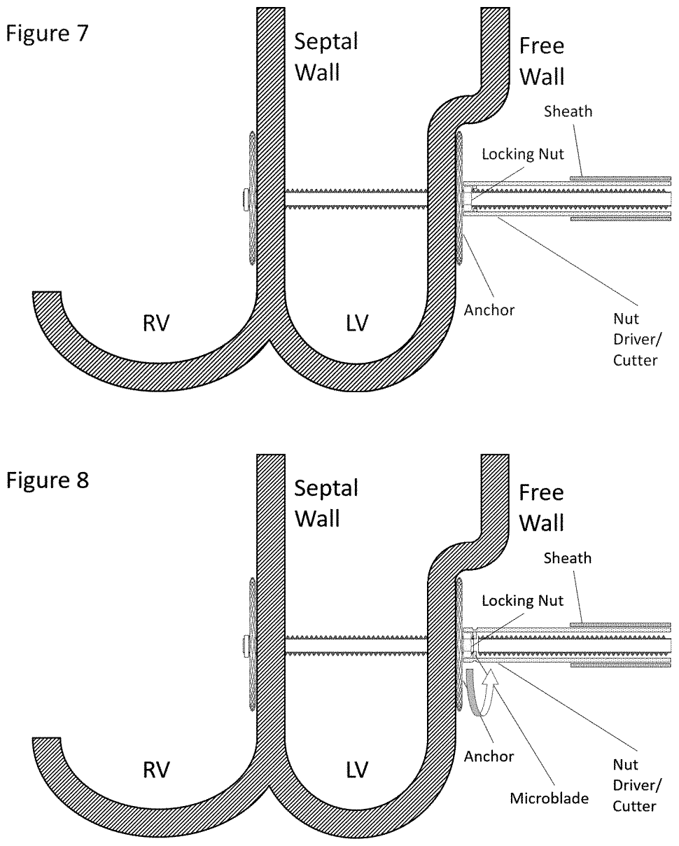

The nut driver/cutter and locking nut can further be advanced over the threaded tether in turn advancing the second anchor in the distal direction and compressing the free wall while the first anchor remains stationary as shown in FIG. 7. The locking nut can be re-positioned in either the distal or proximal direction, allowing the second anchor to be adjusted in distance from first anchor and the effective length of the tether between the walls of the left ventricle to be adjusted as well. Adjustment of the second anchor and reduction in tether length changes the diameter and shape of the left ventricle and alters cardiac function.

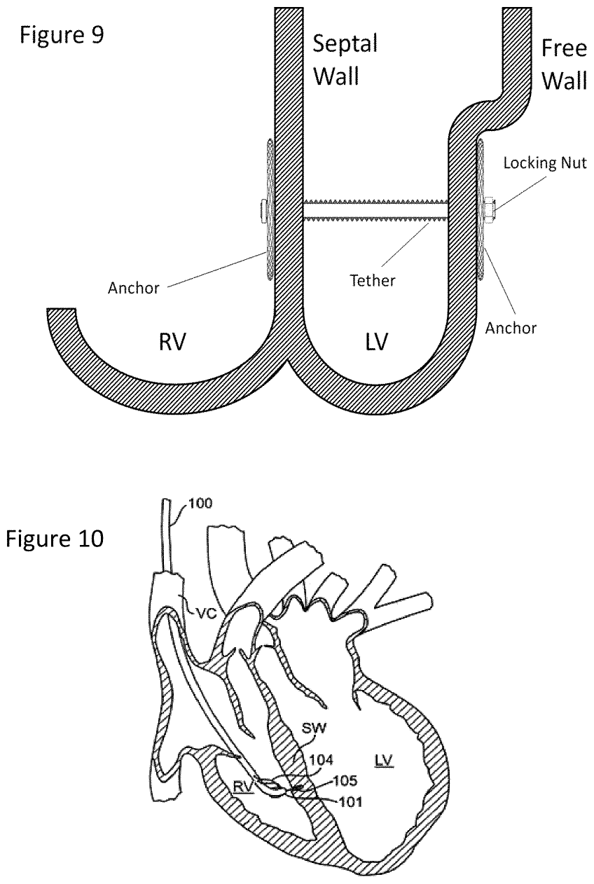

Once the appropriate diameter and shape of the left ventricle is achieved through adjustment of the second anchor, actuation of a microblade on the inner surface of the nut driver/cutter cuts the tether at a location proximal to the locking nut as shown in FIG. 8. The actuation cinches the distal end of the nut driver/cutter and reduces the ID of the microblade to cut into the tether as the nut driver/cutter rotates. Rotation of the nut driver/cutter continues to further cut the tether until it is disconnected from the tether assembly. Cutting the tether severs the tether assembly in the left ventricle from the remainder of the delivery system. Cutting the tether also affixes the second anchor and locking nut in place such that they cannot be re-positioned or adjusted again in the proximal direction. After the tether is cut, the delivery system can be removed leaving tether assembly properly implanted as shown in FIG. 9.

Another embodiment of the endovascular tethering technique according to the present invention is shown in FIGS. 10-17. In this technique, access to the left ventricle LV and delivery of the tether occurs from the right ventricle RV. An approach from within the right ventricle may be preferred for a number of reasons. First, the right ventricle is highly accessible through venous structure that leads into the superior vena cava VC, for example from the right or left jugular veins. Since these veins typically are at a relatively low pressure, bleeding at the access sites is limited, and rather large catheters, cannula and the other like surgical tools can be inserted into the veins in a percutaneous manner. Furthermore, this transcatheter technique permits access to vascular structure without a sternotomy or other open chest surgical access, thereby minimizing trauma to the patient. Additionally, patients are less likely to experience embolic events. Recovery times for the operation also are reduced, due to the minimally invasive nature of such procedures.

Second, delivery through the right ventricle allows for straightforward positioning of the anchors on the ventricular septal wall SW. Such positioning on the septal wall results in good left ventricle bisection, in a manner believed to have minimal negative impact on mitral valve function, and in some instances, a positive impact on mitral valve function and performance. Moreover, delivery through the right ventricle does not involve the free wall of the right ventricle and also does not restrict outflow of the blood from the heart.

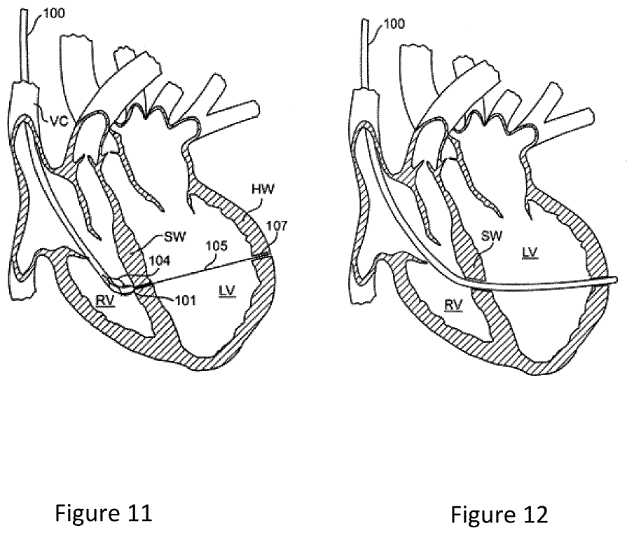

According to the right ventricle delivery technique shown in FIGS. 10-17, a shaped guide device in the form of a delivery catheter 100 is advanced through the vena cava VC and right atrium into the right ventricle RV from an access site, e.g., in the left or right jugular vein. Other access sites, such as, for example, the left or right subclavian vein also are contemplated. As shown in FIG. 10, the catheter has a tip portion 101 configured to be adjustably and variably curved through the use of an adjusting pull-wire 104. The pull-wire is attached to the distal most end of the catheter 101. The pull-wire 104 has a portion that extends exterior the catheter at the distal end of the catheter, and then extends through the catheter to a proximal end of the catheter where it is controlled. As shown in FIGS. 10 and 11, the pull-wire may be an essentially straight wire that, when pulled (or tensioned), causes tip portion 101 to curve. In another embodiment, a pull-wire may take the form of a tether, such as described below with reference to the curved catheter having pull wire. Also in that embodiment, the proximal end of the pull-wire can be pulled and released to thereby cause the distal tip of the catheter to curve and to straighten as desired. Thus, the position of the catheter tip can be curved by adjusting the pull-wire and also advanced or rotated, or both, by advancing or rotating the catheter with respect to the right ventricle and septal wall.

Once the catheter is manipulated to a desired position on the ventricular septum SW, the support wire is advanced to stabilize the tip position, as shown in FIG. 10. A sharpened needle, or guidewire, 105 is then advanced through the lumen in catheter and out of tip portion, piercing the septal wall SW, and extending across the left ventricle chamber LV. Preferably, needle 105 is fabricated of a highly elastic material such as, for example, nickel titanium alloy, which will allow the needle to traverse the bend at the tip of the delivery catheter, and then to straighten out for controlled traversing across left ventricle LV.

Once needle 105 is across the left ventricle chamber, its position is confirmed by TEE, X-Ray, or other visualization techniques, to assure good bisection and avoidance of key mitral valve and other heart structure. Conventional angiography utilizing a “pigtail” catheter. i.e., a dye injection catheter with a loop shape at the distal end, in the left ventricle LV and angiography catheters in one or both coronary artery ostia may also be used to aid in proper positioning of the associated delivery devices in the LV. It also is important to assure that needle 105 will not penetrate or damage any significant coronary vasculature. To assure this, an angiogram may be performed. Preferably, the angiographic image is aligned to a position that looks down the axis of the needle in the portion of the needle which traverses the left ventricle LV. This angle will limit parallax to ensure that if the tip of the needle is not coincident with a significant vessel it will not pierce such vessel. Any small variation in the position of the needle tip can be adjusted by gentle manipulation of the delivery catheter.

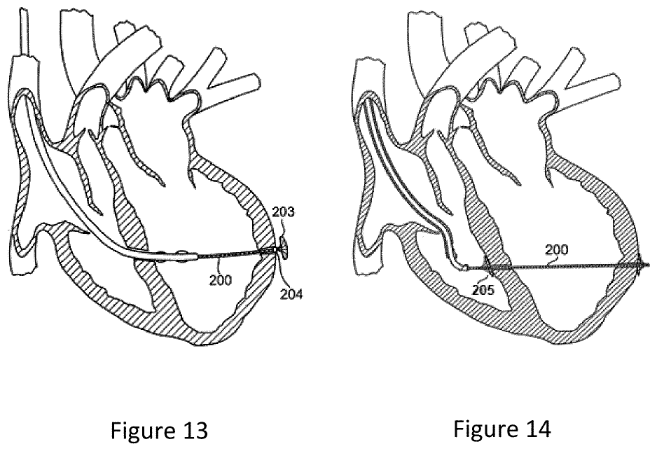

Next, the tether delivery system advances over the guide wire and is inserted through the septal wall, traverses the left ventricle LV, and is inserted through the free wall on the far side of the left ventricle, as shown in FIG. 12. In this method embodiment, the first anchor 203 is then deployed and positioned adjacent to the free wall and the second anchor 205 is deployed and positioned afterwards adjacent to the septal wall as shown in FIGS. 13 and 14. A tether 200 extends between the two anchors.

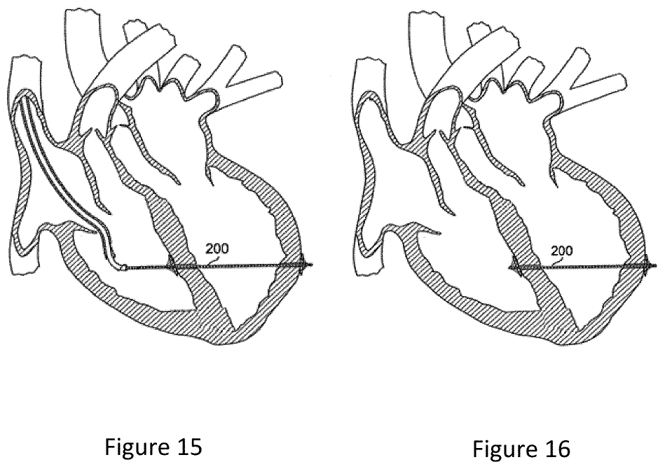

Advancement of the locking nut and nut driver/cutter in the distal direction engages the second anchor in the same direction and compresses the septal wall while the first anchor remains stationary as shown in FIG. 15. The locking nut can be re-positioned in either the distal or proximal direction, allowing the second anchor to be adjusted in distance from first anchor and the effective length of the tether between the walls of the left ventricle to be adjusted as well. Adjustment of the second anchor and reduction in tether length changes the diameter and shape of the left ventricle and alters cardiac function.

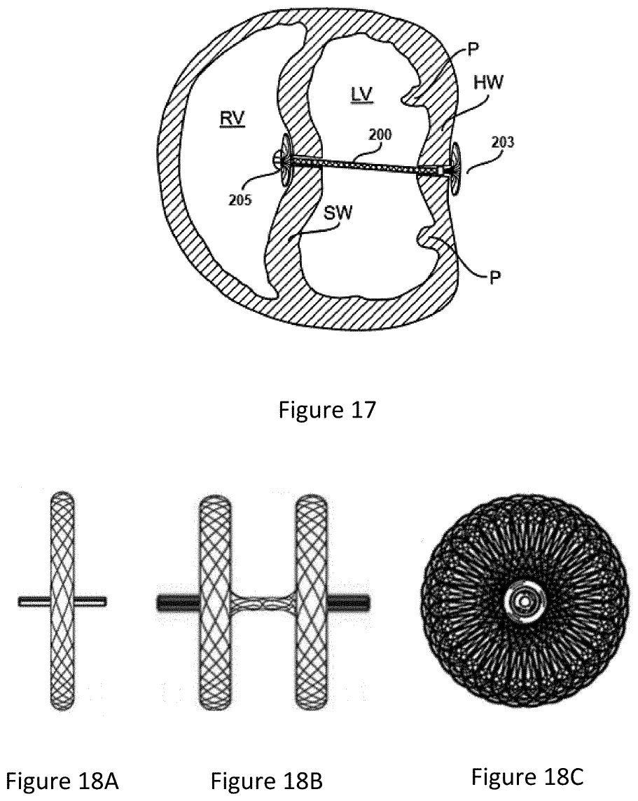

The microblade in the nut driver/cutter is actuated and the tether is severed from the delivery system which is subsequently removed as shown in FIGS. 16 and 17.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A cardiac tether system comprising:
a first anchor having a collapsed delivery configuration and an expanded deployed configuration;
a tether extending proximally from the first anchor, the tether comprising helical threads;
a second anchor having a collapsed delivery configuration and an expanded deployed configuration, the second anchor disposed proximal to the first anchor and being movable with respect to the tether; and
a nut disposed proximal to the second anchor and engaged with the helical threads, the nut being adapted to be rotated with respect to the tether to move the second anchor distally along the tether and to permit the second anchor to move proximally along the tether, the nut being further adapted to prevent movement of the second anchor with respect to the tether in at least one direction when the nut is stationary.

2. The system of claim 1 further comprising a driver adapted to rotate the nut.

3. The system of claim 2 wherein the driver comprises an interior surface shaped to mate with an exterior surface of the nut.

4. The system of claim 2 wherein the driver comprises a cutter adapted to cut a portion of the tether extending proximally from the second anchor.

5. The system of claim 4 wherein the cutter is a microblade actuatable to move from a storage position to a cutting position.

6. The system of claim 1 further comprising a cutter adapted to cut a portion of the tether extending proximally from the second anchor.

7. The system of claim 6 wherein the cutter is a microblade actuatable to move from a storage position to a cutting position.

8. The system of claim 1 wherein the first anchor and/or the second anchor comprises a braided anchor.

9. The system of claim 1 wherein the first anchor and/or the second anchor comprises a laser cut anchor.

10. The system of claim 1 wherein the tether comprises external threads.

11. The system of claim 1 wherein the external threads have a pitch from 56 to 90 threads per inch.

12. The system of claim 1 further comprising a sheath adapted to deliver the first anchor, the tether, the second anchor and the nut endovascularly to an interior location in a patient's heart.

13. The system of claim 1 wherein the first anchor and/or the second anchor comprises a first portion adapted to be disposed on one side of a cardiac wall and a second portion adapted to be disposed on an opposite side of the cardiac wall.

14. A method of modifying a shape of a patient's heart, the method comprising:
placing a first anchor against a surface of a first wall of the heart;
extending a tether proximally from the first anchor to a second wall of the heart;
placing a second anchor against a surface of the second wall;
rotating a nut about the tether to move the second anchor distally along the tether, thereby moving the second wall toward the first wall; and
permitting the nut to remain in place after the rotating step.

15. The method of claim 14 wherein the first wall and the second wall are walls of a left ventricle of the heart.

16. The method of claim 15 wherein the first wall is an exterior wall of the left ventricle and the second wall is a septum of the heart.

17. The method of claim 16 further comprising advancing a sheath into a right ventricle of the heart and moving the first anchor through the septum into the left ventricle.

18. The method of claim 15 wherein the second wall is an exterior wall of the left ventricle and the first wall is a septum of the heart.

19. The method of claim 14 further comprising cutting a portion of the tether extending proximally from the second anchor.

20. The method of claim 19 wherein the step of rotating the nut comprises rotating the nut with a driver, the cutting step comprising actuating a microblade in the driver.
